(19) United States
(12) Patent Application Publication
(10) Pub. No.: US 2024/0074828 A1
(43) Pub. Date: Mar. 7, 2024

(54) MEDICAL DEVICE HOLDING AND MOUNTING SYSTEM

(71) Applicant: Kardion GmbH, Stuttgart (DE)

(72) Inventors: Leon Wenning, Stuttgart (DE); Inga Schellenberg, Stuttgart (DE); Hardy Baumbach, Stuttgart (DE); Thomas Müller, Dresden (DE)

(21) Appl. No.: 18/461,004

(22) Filed: Sep. 5, 2023

Related U.S. Application Data
(60) Provisional application No. 63/374,701, filed on Sep. 6, 2022.

(51) Int. Cl. A61B 50/20; A61B 5/02; A61M 25/00

(52) U.S. Cl. CPC A61B 50/20; A61B 5/02; A61M 25/003; A99Z 99/00

(57) ABSTRACT

An apparatus for holding at least one extracorporeal device of a cardiac-support system includes a receiving unit configured to receive at least one extracorporeal device and a fastening unit configured to fasten the receiving unit to a furniture. The at least one extracorporeal device is connected to an implant device of a cardiac support system via a supply line.

### INCORPORATION BY REFERENCE TO ANY PRIORITY APPLICATIONS

The present application claims priority to U.S. Provisional Application No. 63/374,701, filed Sep. 6, 2022. The above-listed application and any and all other applications for which a foreign or domestic priority claim is identified in the Application Data Sheet as filed with the present application, are hereby incorporated by reference under 37 CFR 1.57, and form a part of this specification for all purposes.

### TECHNICAL FIELD

The disclosure herein generally relates to medical devices. Specifically, the disclosure herein relates to diagnostic, monitoring, or therapeutic devices in medical settings.

### BACKGROUND

In medical practice, there are various procedures requiring diagnostic, therapeutic, or monitoring applications. These applications are typically arranged and managed by highly skilled medical professionals, requiring routine monitoring and power sources. For example, during treatment for cardiogenic shock, a cardiac support system is often used to provide post-operative monitoring and support of the human circulatory system.

### SUMMARY

In one aspect, an apparatus for holding at least one extracorporeal device of a cardiac-support system includes a receiving unit configured to receive at least one extracorporeal device, wherein the at least one extracorporeal device is connected to an implant device of a cardiac support system via a supply line, and a fastening unit configured to fasten the receiving unit to furniture.

The at least one extracorporeal device can be a control unit of the cardiac support system or an energy storage unit of the cardiac support system. The receiving unit can include a coupling element configured to couple the at least one extracorporeal device to the receiving unit. The receiving unit can be configured to receive at least two extracorporeal devices separately from each other. The receiving unit can be detachably connected to the fastening unit. The apparatus can include a cable retention device configured to guide the supply line at least in sections. The apparatus can include a power supply unit configured to provide electrical energy to the at least one extracorporeal device. The apparatus can include a charging cable configured to provide the electrical energy from the power supply unit to at least one extracorporeal device. The power supply unit can be configured to wirelessly transmit the electrical energy to the at least one extracorporeal device. The fastening unit can be configured to connect to the furniture in a form-fitting, non-positive, or material-fitting manner. The apparatus can include a cable guide configured to guide a cable extending between an external power supply and the receiving unit.

In another aspect, a mounting device for coupling a medical device to a holding device includes a connecting device, and a receiving hook attached to the connecting device and configured to couple with a medical device.

The connecting device can include a cable winding section configured to receive a wound cable and a suspension arm configured to couple the connecting device to furniture, wherein the suspension arm is positioned adjacent to the cable winding section. The mounting device can include a charging device configured to provide an output voltage for the medical device. The receiving hook can include an electrical interface configured to be in contact with a corresponding electrical interface of the medical device when the medical device is coupled with the receiving hook. The mounting device can include a charging device, wherein the contact with the electrical interface of the receiving hook and the corresponding electrical interface of the medical device is configured to allow the charging device to provide charge for the medical device. The mounting device can include an adapter member configured to couple to the connecting device and the medical device so that the adapter member is positioned between the connecting device and the medical device, wherein the adapter member is configured to establish electrical communication between the connecting device and the medical device.

In another aspect, a holding device includes a cardiac supporting system that can hold at least one extracorporeal device to a sleeping furniture. The holding device can include a receiving unit shaped to receive at least one extracorporeal device, which can connect to an implant component of the cardiac support system via a supply line, and a fastening unit for fastening the receiving unit to at least one sleeping furniture.

The receiving unit can receive at least one extracorporeal device including, but not limited to, a control unit or an energy storage unit of the cardiac support system. The receiving unit can guide the supply line at least in sections. A power supply unit can provide electrical energy to at least one extracorporeal device. The power supply unit can include a charging cable to provide the electrical energy to at least one extracorporeal device or it can be adapted for wireless transmission of electrical energy.

A fastening unit can be connected to the sleeping furniture in a form-fitting, non-positive, or a material-fitting manner. A holding unit can include a cable guide member, which is shaped to guide a cable connectable to an external power supply. The receiving unit can include a coupling element for connecting at least one extracorporeal device to the holding device and the receiving unit can be detachable from the fastening unit. Optionally, the receiving unit can receive at least two corporeal components separately from each other.

In some embodiments, a holding unit can have at least two separate holding elements, each of the holding elements being shaped to hold at least one or two extracorporeal devices. The fastening unit can have at least two fastening elements for fastening at least two separate holding elements separately from each other to at least one sleeping furniture or two different sleeping furniture.

In another aspect, a suspension device suspends a medical device from a holding device. The suspension device can include a connecting element for mechanically holding various appliances, such as a receiving hook for receiving an appliance, a winding section adjacent to the receiving hook for winding a cable, a suspension arm for hanging the suspension device, and a suspension arm being arranged on a side of the winding section remote from the receiving hook.

In some embodiments, the suspension arm arranged on the side of the winding section can be removed from the receiving hook. A hook unit can have at least one electrical contact which electrically contacts the receiving hook of the suspension device.

A voltage conversion unit can be equipped and adapted to provide an output voltage. The receiving hook can include an electrical interface for charging the cable, which is adapted to receive an electrical interface of the medical device to charge the cable of the device with the output voltage. Securing elements in a connected state of the device can include at least one clip, magnet, rubber band, hook, snap fastener, hook-and-loop element and/or strap. An adapter member can electrically connect the connecting member to the device when a device terminal and a connecting terminal of the connecting member cannot be coupled.

A medical device can have a control unit and a storage unit for storing electrical energy. The control unit and the storage unit can be configured to couple together by means of a plug connection unit, a spring unit, a latching unit, a slide-in unit, or a magnetic coupling unit. The memory unit can be integrated into the control unit. In some embodiments, the medical device can be adapted to communicate with other devices by means of a wireless connection.

A medical device can be suspended in the receiving hook that can output a charging voltage (or current) to the medical device.

### BRIEF DESCRIPTION OF THE DRAWINGS

FIG. 1 is a cross sectional view of a heart showing a mechanical circulatory support (MCS) device mounted on a catheter and positioned across an aortic valve via a femoral artery access.

FIG. 2 schematically illustrates an access pathway from the femoral artery to the left ventricle.

FIG. 3 is a side view of a catheter delivery system.

FIG. 4 is a schematic side view of a holding device attached to a piece of furniture.

FIG. 5 is a perspective view of an example mounting device for hanging a medical device on a holding device.

FIG. 6 is a perspective view of an example mounting device.

FIG. 7 is a schematic side view of an example mounting device.

FIG. 8 is a schematic front view of an example mounting device.

FIG. 9 is a schematic front view of another example of a mounting device.

FIG. 10 is a perspective view of an example adapter element for a hanging device.

FIG. 11 is a perspective view of another example of an adapter element.

FIG. 12 is a schematic front view of an example medical device.

FIG. 13 is a schematic front view of another example medical device.

FIG. 14 is a schematic front view of yet another example medical device.

FIG. 15 is a schematic front view depicting securing of the medical device of FIG. 14.

FIG. 16 is a perspective view of an example slide-in unit of a mounting device.

FIG. 17 is a perspective view of another example slide-in unit of a mounting device.

FIG. 18 is a schematic top view of another example slide-in unit of a mounting device.

FIG. 19 is a perspective view of another example slide-in unit of a mounting device.

FIG. 20 is a flow chart of an example method for operating a mounting device according to a design example.

FIG. 21 is a block diagram of an example fixture.

FIG. 22 is a schematic representation of operating an example medical device.

FIG. 23 is a schematic view of an example medical device.

FIG. 24 is a schematic view of another example of a medical device.

FIG. 25 is a schematic view depicting operation of an example medical device.

### DETAILED DESCRIPTION

Percutaneous coronary intervention (PCI) is a minimally invasive procedure that requires cardiac catheterization to place a stent to open coronary arteries that are narrowed or blocked by the buildup of atherosclerotic plaque. After such a procedure, a cardiac support system is often used to provide post-operative monitoring and support of a patient's circulatory system. Such a cardiac support system can often include an extracorporeal monitoring device connected to a circulatory support system via a cable. The extracorporeal monitoring device is often attached to at least one piece of furniture (for example, sleeping furniture). However, lack of proper support for the monitoring device can result in damage to the monitoring device and injury to the patient. For example, patients may be at risk of falling by tripping over the cable. Further, the maintenance of unsupported devices can be costly and reduce the quality of patient care by causing discomfort and pain. As such, a safe and effective holding system along with a mounting device (for example, a suspension device) for an extracorporeal device can be beneficial.

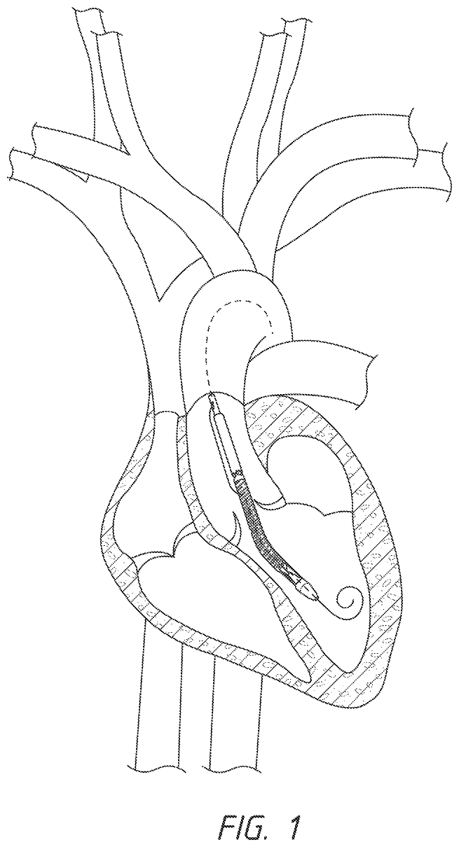

FIG. 1 shows a cross-sectional view of a heart showing a catheter extending into a left ventricle of the heart during, for example, a PCI procedure. As shown in FIG. 1, a mechanical circulatory support (MCS) device can be mounted at a distal end of the catheter. An inlet tube portion of the MCS device can extend across an aortic valve of the heart.

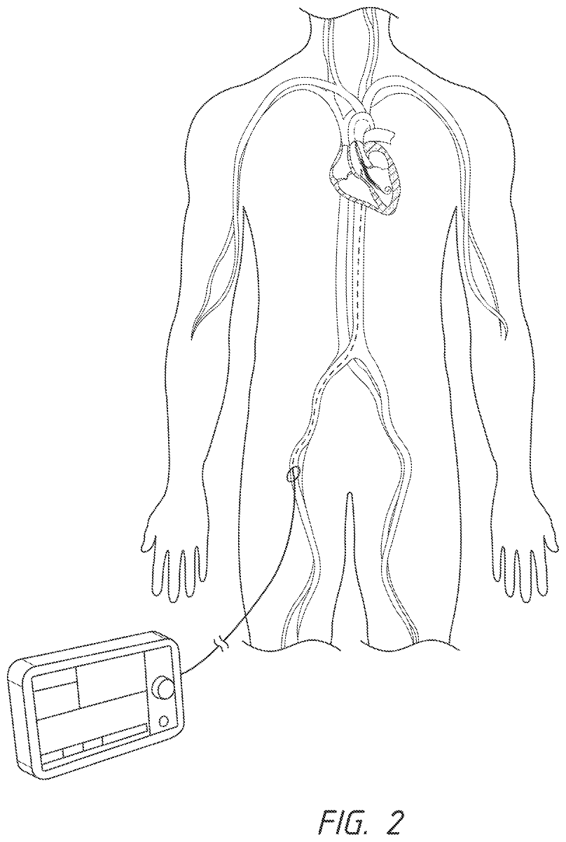

FIG. 2 shows an example access route for an example catheter for, for example, PCI procedures. As shown in FIG. 2, the catheter can be inserted via a femoral artery and routed towards the heart.

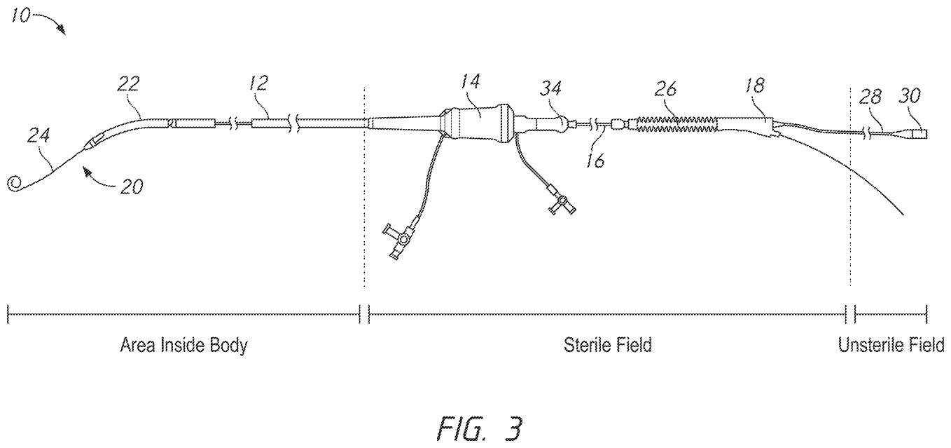

FIG. 3 shows the catheter system 10. The catheter system 10 can include an introducer sheath 12 having a proximal introducer hub 14 with a central lumen for axially movably receiving a shaft 16. The shaft 16 can be coupled to and facilitate movement (for example, towards the heart) of an MCS device. The shaft 16 can extend between a proximal hub 18 and a distal end 20. The proximal hub 18 may be provided with a controller (for example, an integrated microcontroller) or a memory storage device for device identification and tracking of running time (for example, of the MCS device), which could be used to prevent overuse (for example, of the MCS device) and reduce the likelihood of excessive wear or other malfunctions associated with overuse. The controller or the memory device could disable the device (for example, the MCS device), for example, to prevent usage of a used device (for example, a used MCS device such as a pump). The controller may generate and display information about the device (for example, the MCS device) or messages about its usage.

A pump 22 can be disposed in a distal region of the shaft 16. The catheter system 10 can include at least one central lumen for axially movably receiving a guide wire 24. The proximal hub 18 can include an infection shield 26. A proximal cable 28 can extend between the proximal hub 18 and a connector 30 for releasable connection to a control system for the catheter system 10. The control system for the catheter system 10 can be positioned outside of the sterile field. The control system for the catheter system 10 can drive the pump 22. The control system for the catheter system 10 may include an extracorporeal device, such as batteries, which can be attached to at least one sleeping furniture (for example, a bed).

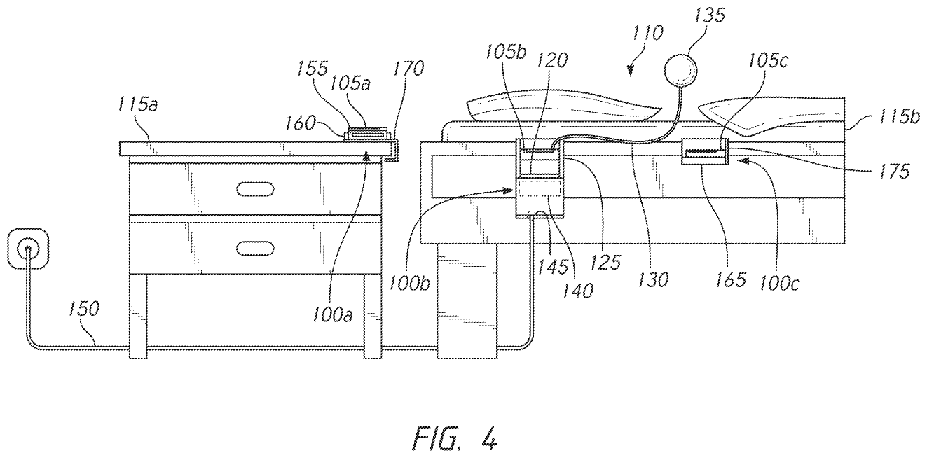

FIG. 4 shows a schematic representation of holding devices 100a, 100b, 100c. Each of the holding devices 100a, 100b, 100c, is configured to hold at least one extracorporeal device of a cardiac support system 110. As shown in FIG. 4, the holding devices 100a, 100b, and 100c can hold extracorporeal devices 105a, 105b, and 105c, respectively. The holding devices 100a, 100b, 100c can be attached to furniture 115a, 115b as shown in FIG. 4. In some embodiments, the furniture can be sleeping furniture. The holding devices 100a, 100b, 100c can each include a mounting unit or holding unit or receiving unit 120 and an attachment unit or fastening unit 125. The receiving unit 120 can receive the at least one extracorporeal device 105a, 105b, 105c. The at least one extracorporeal device 105a, 105b, 105c can be connected to an implant component 135 of the cardiac support system 110 via a supply line 130. The fastening unit 125 can attach the receiving unit 120 to the at least one furniture 115a, 115b.

The holding devices 100a, 100b, 100c can be used as a holder for a nightly power supply, for example, of a cardiac support system 110 (for example, an MCS system or a ventricular assist device (“VAD”) system) for patients with, for example, heart failure. A bed frame and a bedside table are shown here as examples of the furniture 115a, 115b. In other embodiments, the furniture 115a, 115b can be other types of furniture or an aid in a sleeping environment of a patient with, for example, the cardiac support system 110. For example, in some embodiments, the furniture 115a, 115b can include a sofa bed or a hospital bed frame. The implant component 135 can include a heart pump, for example. The implant component 135 can be connected to at least one of the extracorporeal devices 105a, 105b, 105c via the supply line 130. The supply line 130 can be a cable line that can connect the implant component 135 with the extracorporeal devices 105a, 105b, 105c through a patient's skin, also referred to as a “driveline”.

FIG. 4 shows two examples of the holding devices, the receiving unit 120 and the fastening unit 125, each being integrated into a single-piece device in one example, and the receiving unit 120 and the fastening unit 125 each being two-piece devices in another example. Further details of the receiving unit 120 and the fastening unit 125 are provided herein.

In some implementations, the receiving unit 120 can receive a control unit of the cardiac support system 110 as the at least one extracorporeal device 105a, 105b, 105c. Additionally or alternatively, the receiving unit 120 can receive an energy storage unit of the cardiac support system 110 as the at least one extracorporeal device 105a, 105b, 105c. The energy storage unit can include one or more rechargeable batteries. The receiving unit 120 can at least partially enclose the control unit (and/or the energy storage unit) of the cardiac support system 110. If a control unit of the cardiac support system 110 is connected to an energy storage unit of the cardiac support system 110 via a cable, the receiving unit 120 can receive both the control unit and the energy storage unit in the connected state.

The receiving unit 120 can guide the supply line 130 at least in sections. For example, the receiving unit 120 can guide the supply line 130 at least in sections into or along the receiving unit 120. This can prevent the supply line 130 from being disconnected from at least one extracorporeal device 105a, 105b, 105c, including the control unit. The supply line 130 can include a strain relief that can provide additional comfort. A strain relief can be positioned between the control unit and the supply line 130. For example, a spiral cable can be used as a strain relief.

The holding devices 100a, 100b, 100c can each include a power supply unit 140 that can supply electrical energy to the at least one extracorporeal device 105a, 105b, 105c. For example, the power supply unit 140 can function as the nightly power supply for the cardiac support system 110 when the at least one extracorporeal device 105a, 105b, 105c is arranged (for example, stowed or stored) in the receiving unit 120. For instance, the power supply unit 140 can be connected to an external power source (for example, a standard wall plug) or another energy source. As such, at least one extracorporeal device 105a, 105b, 105c can thus be fixed (for example, stowed or stored) in the holding devices 100a, 100b, 100c and receive electrical energy drawn from, for example, an external power source.

The power supply unit 140 can include a charging cable. For example, the charging cable can be connected to at least one extracorporeal device 105a, 105b, or 105c, to accommodate the receiving unit 120, or to link an external power connection (for example, a standard wall plug) via the power supply unit 140 or a main plug. Additionally, the charging cable can connect to additional extracorporeal device if a control unit (for example, of the cardiac support system 110) and an energy storage unit (for example, of the cardiac support system 110) are accommodated (for example, stowed or stored) in the receiving unit 120. In some implementations, the power supply unit 140 can provide wireless transmission of electrical energy. For example, the energy supply unit 140 can provide inductive energy transmission to charge the control unit (for example, of the cardiac support system 110) or batteries of the energy storage unit (for example, of the cardiac support system 110).

In some implementations, the receiving unit 120 can include a cable guide element 145 that can guide a cable 150 that can be connected to an external power supply (for example, a standard wall plug). For example, the cable guide element 145 can be a guide ring. The cable guide element 145 can shorten the length of the cable 150 and can reduce the risk of tripping over the cable 150.

The receiving unit 120 can include a coupling element 155 for connecting the extracorporeal devices 105a, 105b, 105c with the holding devices 100a, 100b, 100c. For example, the coupling element 155 can be a retaining lug or a clip closure. If the holding devices 100a, 100b, 100c include the power supply unit 140, the coupling element 155 can be formed as a docking mechanism for coupling the at least one extracorporeal device 105a, 105b, 105c to the power supply unit 140. The coupling element 155 can simplify the positioning of the extracorporeal devices 105a, 105b, 105c (or coupling of the extracorporeal devices 105a, 105b, 105c to the holding devices 100a, 100b, 100c), particularly when the extracorporeal devices 105a, 105b, or 105c are placed in the holding devices 100a, 100b, or 100c in dimly lit environments (for example, at night).

The receiving unit 120 can be separately connected to the fastening unit 125. This can allow the extracorporeal device (for example, the extracorporeal devices 105a, 105b, 105c) to be arranged (for example, stowed) in the receiving unit 120 without the receiving unit 120 being connected to the fastening unit 125. Additionally, it is possible to use the receiving unit 120 independently of the fastening unit 125 to mount (for example, receive) at least one extracorporeal device 105a, 105b, 105c and transport the extracorporeal device in a carrying system while it is arranged (for example, stowed) in the receiving unit 120. Additionally or alternatively, a fastening unit 125 can be attached to each of the furniture 115a, 115b, and a receiving unit 120 can be connected to the fastening unit 125.

The fastening unit 125 can be connected (for example, removably coupled) to the furniture 115a, 115b. For example, the fastening unit 125 can be connected to the furniture 115a, 115b in a friction-locked (or friction-fit) manner. In some examples, the fastening unit 125 can include a lever mechanism with a toggle lever in a swing lock, a screw mechanism, a clamp, and/or an adhesive surface to connect the fastening unit 125 to the furniture 115a, 115b by using an adhesive.

In some implementations, the receiving unit 120 can accommodate more than one extracorporeal device 105a, 105b, 105c. This can allow disconnection of one of the extracorporeal devices (for example, the extracorporeal devices 105a, 105b, 105c) from the supply line 130, for example to replace a battery, while the other extracorporeal device(s) remain connected to the supply line 130.

In some implementations, the receiving unit 120 can include at least two separate receiving elements 160, 165. Alternatively, the receiving unit 120 can include either the receiving element 160 or the receiving element 165. Each of the receiving elements 160, 165 can be dimensioned or shaped to accommodate one of the extracorporeal devices 105a, 105b, 105c. The fastening unit 125 can include at least two fastening elements 170, 175 for fastening the receiving elements 160, 165 independently from each other to the furniture 115a, 115b.

The holding devices 100a, 100b, 100c can enable a guided cable connection, in which a cable connection from the extracorporeal devices 105a, 105b, or 105c to a main adapter or to a socket is advantageously not loosely located between the furniture 115a, 115b and the socket. Additionally, the holding devices 100a, 100b, 100c, being attached to the furniture 115a, 115b, can prevent the extracorporeal devices 105a, 105b, 105c from causing strangulation or pain when the patient moves or turns (for example, during sleep) and causes tension on the supply line 130 connecting the cardiac support system 110 attached to the patient and the extracorporeal devices 105a, 105b, 105c. Furthermore, the holding devices 100a, 100b, 100c can prevent damage to or disconnection of the extracorporeal devices 105a, 105b, 105c (for example, a control unit or batteries), which would otherwise be stored on a bed or on a bedside table and could fall out of the bed or the bedside table when the patient moves. By improving patient mobility in various situations, the system described herein can also improve patient's sleep hygiene. The power supply unit 140 of the holding devices 100a, 100b, 100c can allow the extracorporeal devices 105a, 105b, 105c to remain close to the furniture 115a, 115b without needing to be connected to an external power source (for example, a standard wall plug), which is convenient for the patients.

When the holding devices 100a, 100b, 100c for the extracorporeal devices 105a, 105b, 105c of the cardiac support system 110 are attached to a bed frame or other furniture near the patient, they can prevent risks of falling and possible damage to the extracorporeal devices 105a, 105b, 105c. The extracorporeal devices 105a, 105b, 105c of the cardiac support system 110 can include a control unit (for example, a processor) and a battery, which can be fixed and recharged in the holding devices 100a, 100b, 100c. Routing the cable 150 between an external power source (for example, a standard wall plug) and the holding devices 100a, 100b, 100c coupled to the furniture 115a, 115b can allow for a reduced cable length and increase in patient safety. A simple docking mechanism of the holding devices 100a, 100b, 100c can allow for easier storing (or stowing) and charging of the extracorporeal devices 105a, 105b, 105c while being stored in the holding devices 100a, 100b, 100c. The fastening unit 125 can allow moving and fastening of the extracorporeal devices 105a, 105b, 105c in different locations while being stored (or stowed) in the holding devices 100a, 100b, 100c. Some other locations where the holding devices 100a, 100b, 100c stowing the extracorporeal devices 105a, 105b, 105c can be attached to include a bed rail, a ceiling, an IV drug stand, and a wall plate.

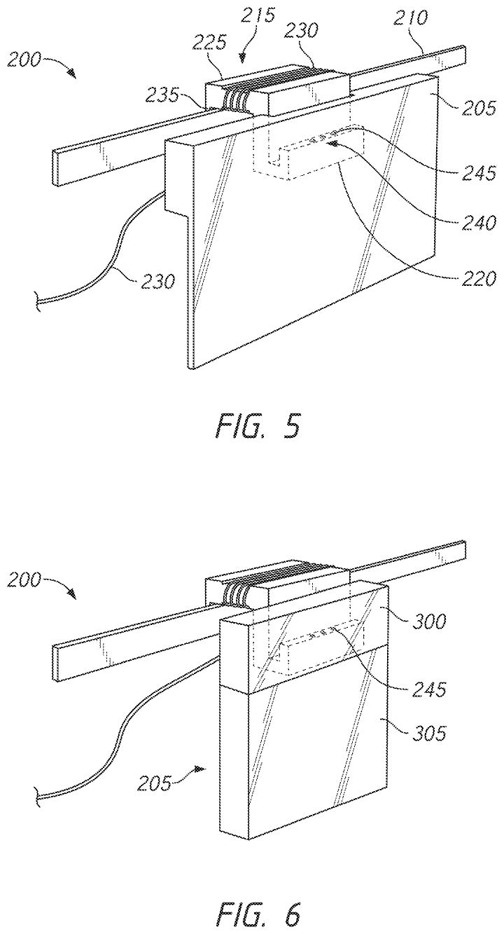

FIG. 5 shows a perspective view of an example mounting device 200 (for example, a suspension device) for suspending or hanging (for example, storing or stowing) a medical device 205 via a holding device 210 on, for example, furniture. The medical device 205 can be a tablet, for example. The medical device 205 can be a single-piece device or multi-piece device. The holding device 210, for example, can be a standard rail that can be attached to furniture (for example, a bed). The mounting device 200 can include a connecting device 215 which can be shaped to receive the medical device 205 and can couple to the holding device 210. Additionally, the connecting device 215 can receive power from an external power source and route the power to the medical device 205. As shown in FIG. 5, the medical device 205, can include or be coupled to a hook that can suspend the medical device 205 on the mounting device 200.

The connecting device 215 can include a mounting or receiving hook 220 for mounting (for example, suspending) the medical device 205, a winding section 225 adjacent to the hook 220 for routing (for example, winding) a cable 230, and a suspension arm 235 for suspending the mounting device 200 (for example, removably coupling the mounting device 200 to the holding device 210). The suspension arm 235 can be located on a side of the winding section 225 facing away from the hook 220.

In some implementations, the hook 220 can be approximately J-shaped. Optionally, the mounting device 200 can include a converter (for example, DC/DC converter or AC/DC converter) that can receive an input current, for example, via the cable 230, and convert the input current (for example, AC) to an output current (for example, DC) that can be transmitted to, for example, the medical device 205. The hook 220 can include an electrical interface 240 for charging the medical device 205 with the output current. The converter (for example, an AC/DC converter) can be housed in an interior of the winding section 225, so that the mounting device 200 can function as a power supply unit. The electrical interface 240 can include at least one electrical contact 245. In some examples, the electrical interface 240 can be a cable socket (for example, USB interface).

The mounting device 200 can include at least one safety element that can secure a mechanical connection between the medical device 205 and the hook 220. For example, the securing element can include at least one of a clip, a magnet, a rubber band, a hook, a snap fastener, a hook-and-loop fastener and a strap to secure the medical device 205 to prevent it from falling (for example, off of the hook 220).

The connecting device 215 described herein can function as a secure, space-saving storage and a charging dock for the medical device 205 at a location proximate to a patient. Since the connecting device 215 can provide charge for the medical device 205, the medical device 205 can, in turn, provide power for any medical devices (for example, the cardiac support system 110) attached to the patient. Moreover, the connecting device 215 can provide improved cable management for the medical device 205 and other devices (for example, the cardiac support system 110). The electrical interface 240 can allow users (for example, care providers) to couple devices (for example, the medical device 205) directly to the connecting device 215 without plugging in additional cables.

The holding device 210 can offer a safe, space-saving, variable and universal possibility for suspension. The cable 230 can be connected to a power plug (for example, a standard wall plug) or a cable extending between the connecting device 215 to a control unit (for example, the medical device 205 or the extracorporeal devices 105a, 105b, 105c) and can be wound up at the winding section 225. For example, by using the holding device 210 and the connecting device 215 as a support, medical devices (for example, the extracorporeal devices 105a, 105b, 105c) can be attached in convenient locations without use of any additional brackets or stands. Furthermore, safety for the patient can be increased as the risk of a fall of the device 205 can be significantly reduced.

The connecting device 215 can be removably or permanently attached to the holding device 210. Optionally, the connecting device 215 can be secured against slipping out (for example, detaching from the holding device 210) by using a safety element. The safety element can include a clip, magnets, or any other suitable features to safely attach the connecting device 215 to the holding device 210. The medical device 205 (for example, the extracorporeal devices 105a, 105b, 105c) can include a control unit and a battery, and can be coupled to the connecting device 215. As described herein, the medical device 205 can be suspended over at least a portion of the connecting device 215. Additionally or alternatively, the medical device 205 can be connected to the connecting device 215 via at least one electrical contact 245 or via a cable (for example, a USB cable) extending between and connected to the medical device 205 and the connecting device 215.

In some implementations, other devices such as, but not limited to, magnets, rubber bands, hooks, belts, and so forth can be used to couple the connecting device 215 to the holding device 210. Such devices can be used to help maintain the coupling between the medical device 205 and, for example, the hook 220 of the connecting device 215.

FIG. 6 shows another example of the mounting device 200 stowing a medical device (for example, the medical device 205) having multiple components. The medical device 205 can include a control unit 300 and a storage unit 305 for, for example, storing electrical energy. The control unit 300 and the storage unit 305 can be permanently fixed to each other or detachable from each other. This is possible by means of a spring unit, a latching unit, a slide-in unit and/or a magnetic coupling unit. The control unit 300 can be dimensioned and shaped to removably couple with the hook 220 of the mounting device 200.

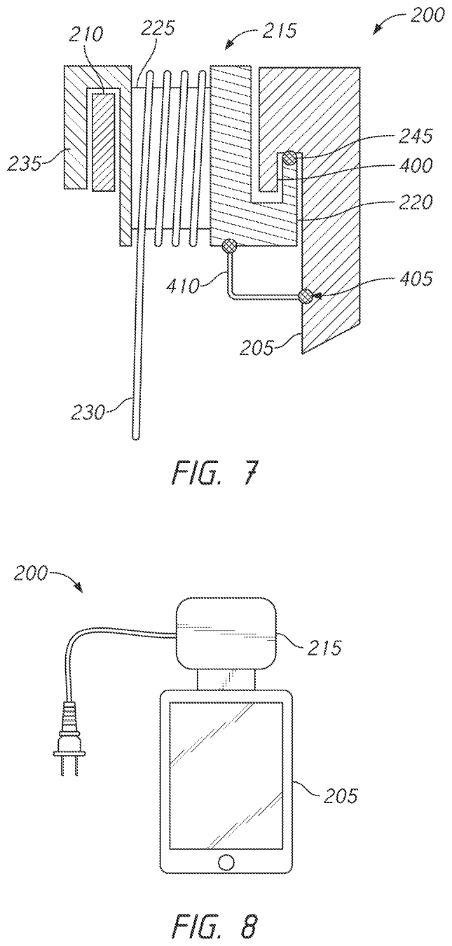

FIG. 7 shows a schematic side view of an example of the mounting device 200. The medical device 205 can include a hook 400 that can engage (for example, couple with) the corresponding hook 220. The medical device 205 and the connecting device 215 can be connected to each other via at least one electrical contact 245 or via a cable 410 extending between and connected to the connecting device 215 and a cable connection 405 of the medical device 205. As shown in FIG. 7, the cable 230 can be wound up around the winding section 225 of the connecting device 215. Additionally, a connecting cable 410 between the medical device 205 and the connecting device 215 can be wound up around the winding section 225.

As shown in the example in FIG. 7, the hook 220 can be approximately J-shaped and can interlock with the hook 400 to allow coupling between the connecting device 215 and, for example, the medical device 205 or extracorporeal devices 105a, 105b, 105c. The suspension arm 235 of the connecting device 216 can engage (for example, wrap around) the holding device 210.

FIG. 8 shows a schematic representation of another example of the mounting device 200 coupled to the medical device 205. As shown in FIG. 8, the medical device 205 can be suspended from the connecting device 215 via a direct connection. The direction connection between the connecting device 215 and the medical device 205 can be made between, for example, a male connector on the connecting device 215 and a corresponding female connector on the medical device 205. The coupling between the male connector and the corresponding female connector can maintain the coupling between the connecting device 215 and the medical device 205. In some examples, magnets, detents, latches, or other suitable features can be used to help maintain the coupling between the connecting device 215 and the medical device 205. The coupling between the connecting device 215 and the medical device 205 can allow transmission of electronic signals between the connecting device 215 and the medical device 205.

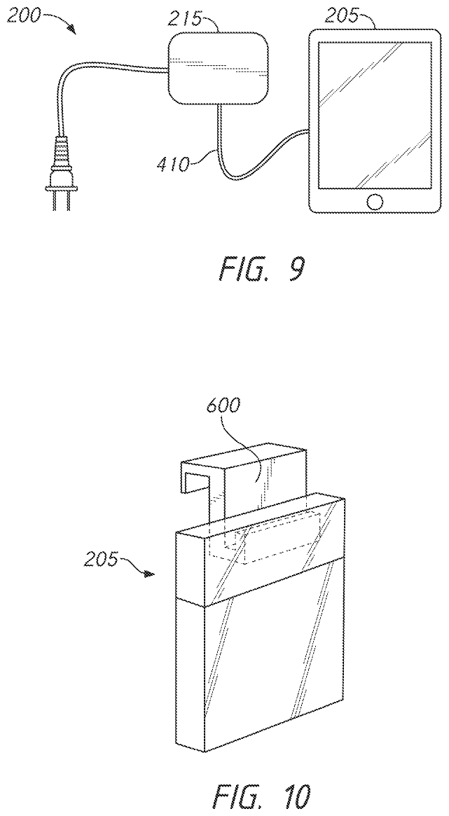

FIG. 9 shows a schematic representation of another example of the mounting device 200. As shown in FIG. 9, the medical device 205 and the connecting device 215 can be connected to each other via the cable 410.

FIG. 10 shows a perspective view of an example adapter 600. The adapter 600 can include two J-shaped hooks extending in two opposite directions. The medical device 205 can be connected (for example, stowed) to the adapter 600. Optionally, the adapter 600 can be coupled to the hook 220 of the connecting device 215. Optionally, the adapter 600 can be directly coupled to the holding device 210, separate from the connecting device 215. If connectors of the medical device 205 and the connecting device 215 are not compatible, the adapter 600 can be used (for example, one side coupled to the connecting device 215 and another side coupled to the medical device 205) to establish electrical connection between the connecting device 215 and the medical device 205 while allowing the medical device 205 to be suspended off of the connecting device 215.

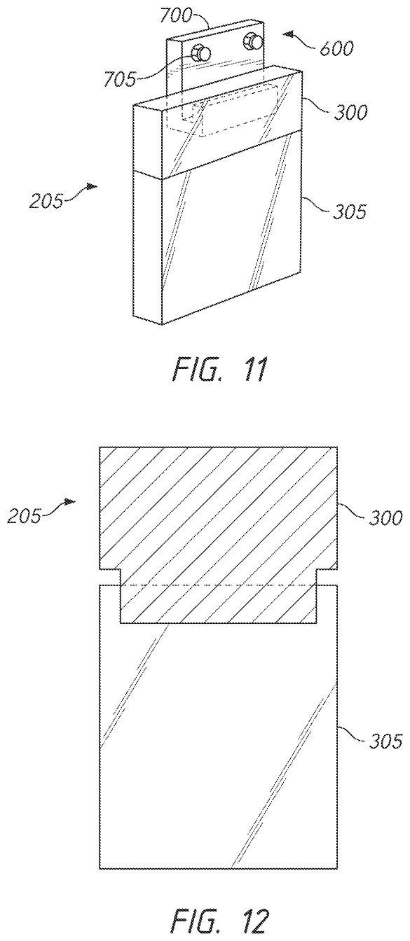

FIG. 11 shows a perspective view of another example, of the adapter 600. As shown in FIG. 11, the adapter 600 can include a single J-shaped hook. The adapter 600 can include a fastening end 700 that can include at least one opening 705. The opening 705 can be used to attach the adapter 600 to, for example, a mounting wall, plate, bar, and so forth.

FIG. 12 shows a schematic front view of an example of the medical device 205. The device 205 can include the control unit 300 and the storage unit 305 as described herein. The control unit 300 can be T-shaped and a narrower portion of the control unit 300 can, for example, be inserted into a corresponding opening of the storage unit 305 to create a removable connection between the control unit 300 and the storage unit 305.

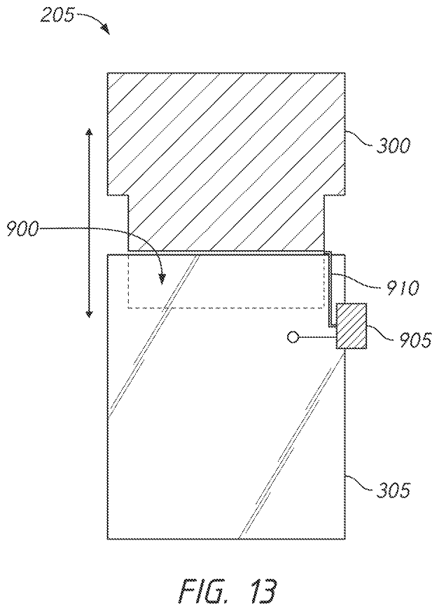

FIG. 13 shows a schematic front view of the example medical device 205 of FIG. 12. In FIG. 13, the control unit 300 and the storage unit 305 are decoupled from each other. The storage unit 305 can include a recess 900 to accommodate the control unit 300 (for example, a narrower portion of the control unit 300 as described herein). The storage unit 305 can include a button 905 that can be used to secure a body of the control unit 300 to the storage device 305. The button 905 can actuate a snap-in hook 910 that can engage the control unit 300 to secure the control unit 300 to the storage unit 305.

In some implementations, the recess 900 of the storage unit 305 can include a spring mechanism that can be moved between an unactuated position and an actuated position. For example, when a portion of the control unit 300 is inserted into the groove 900 of the storage unit 305, the spring mechanism can move from the unactuated position to the actuated position. A locking interface (for example, a hook or a latch) can hold the control unit 300 in place after insertion into the groove 900. Actuation of the button 905 (for example, pressing) can release the locking interface and allow the spring mechanism to move from the actuated position to the unactuated position, pushing the control unit 300 out of the groove 900.

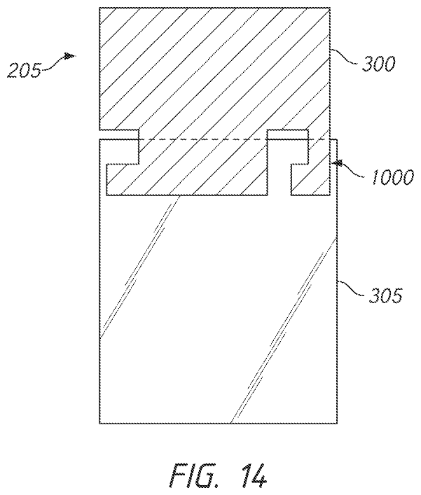

FIG. 14 shows a schematic front view of another example of the medical device 205. The control unit 300 can include an edge 1000 facing the storage unit 305 in a hook-like shape. The edge 1000 can engage a counterpart (for example, a groove or an opening) formed on the storage unit 305.

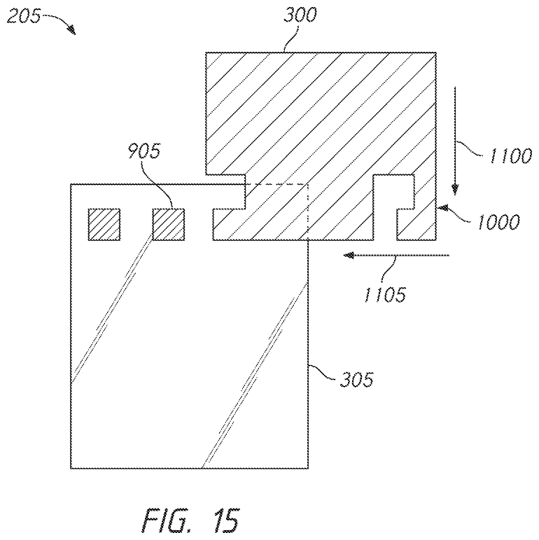

FIG. 15 shows another schematic front view of the medical device 205 of FIG. 14. The storage unit 305 of the medical device 205 can include at least one coupling element 905 that can be used to couple the storage unit 305 (or the medical device 205) to the mounting device 200. The coupling element 905 can be at least one magnet that can interact with (for example, couple with) a counterpart element of the control unit 300. To couple the control unit 300 and the storage unit 305 to each other, these two components can initially at least partially offset each other along a first direction 1100 and then can be pushed in a second direction 1105. The first direction 1100 and the second direction 1105 may be orthogonal to each other. That way, the coupling elements 905 of the storage unit 305 and the counterpart element of the control unit 300 can be secured to each other.

In this embodiment, the first direction 1100 can represent a vertical movement of the control unit 300 into or towards the storage unit 305. The second direction 1105 can represent a horizontal movement of the control unit 300 across a width of the storage unit 305. For example, the edge 1000 can include a vertical portion (for example, extending away from the body of the control unit 300) and a horizontal portion (for example, extending across the body of the control unit 300). The horizontal portion can be inserted into, for example, a corresponding groove formed on the body of the storage unit 305. In other words, the control unit 300 can be inserted from above and then laterally across the storage unit 305. The control unit 300 can be pushed laterally until it can no longer be pushed laterally. Optionally, the control unit 300 and the edge 1000 can be secured using the coupling element 900 described herein.

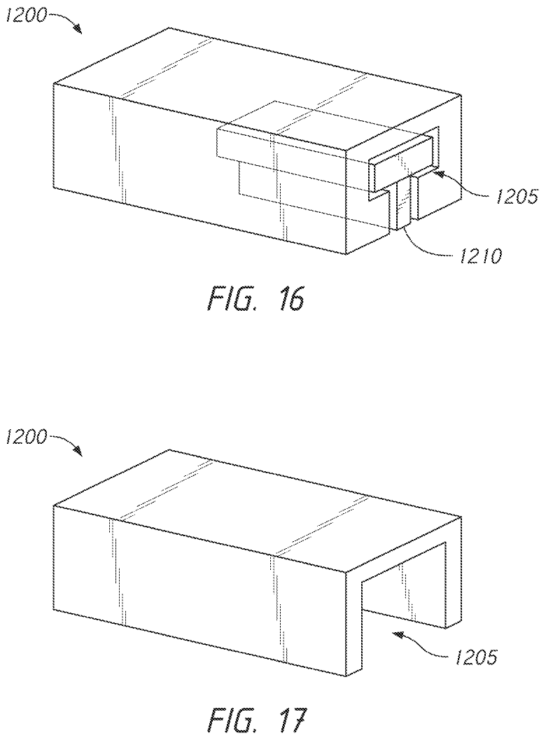

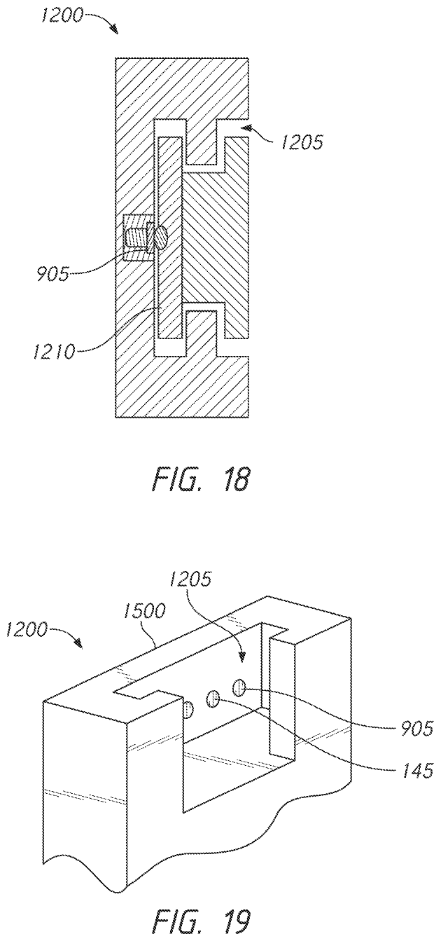

With reference to FIGS. 16-19, various examples of a slide-in unit 1200 for the mounting device 200 are disclosed. The slide-in unit 1200 can include a groove 1205 that can receive a corresponding insertion 1210 of, for example, the medical device 205. The mounting device 200 can include the slide-in unit 1200 in addition to the hook 220.

Optionally, the cross-sectional shape of the groove 1205 can be square, rectangular, or any other suitable shapes.

Optionally, the insertion 1210, for example, of the medical device 205 can be H-shaped. The groove 1205 of the slide-in unit 1200 can have a cross-sectional shape that corresponds to a negative form of the shape of the insertion element 1210. In some examples, the slide-in unit 1200 can include at least one coupling element 905. The coupling element 905 can be a magnet can help maintain coupling between the slide-in unit 1200 and the insertion element 1210.

The groove 1205 can include a wall 1500 forming the groove 1205 having an opening 1505 on one side. At least one of the coupling element 905 and/or at least one of the electrical contact 145 can be arranged on the wall 1500 opposite the opening 1505.

FIG. 20 shows a flowchart of a method 1600 for operating a mounting device (for example, the mounting device 200). At block 1605, the medical device (for example, the medical device 205) is stowed (for example, suspended) via the pick-up hook (for example, the hook 220). While stowed, electrical contacts of the medical device 205 and corresponding electrical contacts (for example, the electrical contacts 245) of the mounting device 200 can be in contact. At block 1610, the mounting device 200 detects presence of the medical device 205 (for example, via the contact between electrical contacts of the medical device 205 and corresponding electrical contacts of the mounting device 200) and provide charge to the medical device 205.

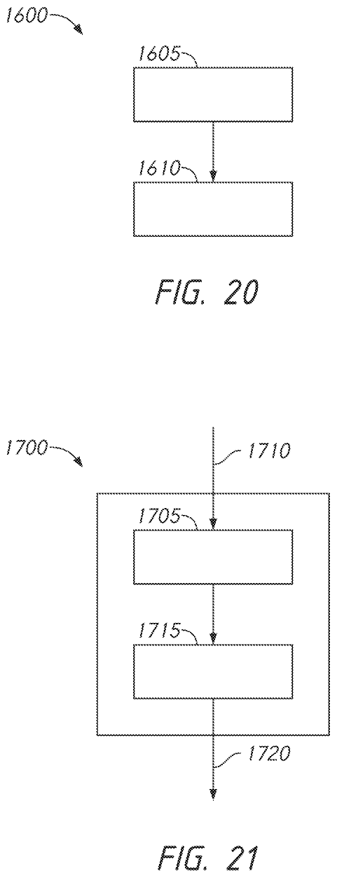

FIG. 21 shows a block diagram of a fixture 1700. The fixture 1700 can include an acquisition unit 1705 that can acquire (for example, detect) the medical device (for example, the medical device 205) suspended (or coupled) in the recording hook (for example, the hook 220) via an acquisition signal 1710. The fixture 1700 can include an output unit 1715 that can control the output of the charging voltage (or charging current) to the medical device (for example the medical device 205) by generating an output signal 1720.

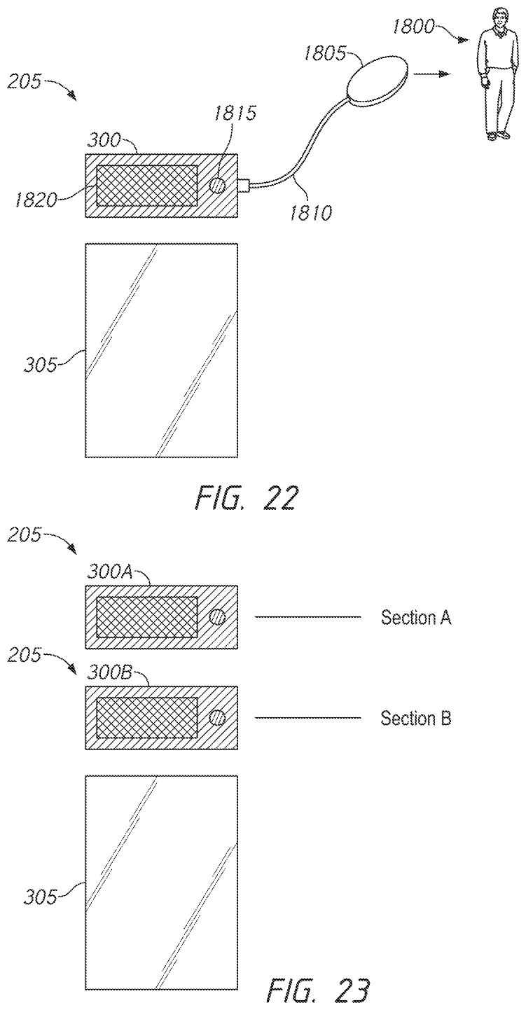

FIG. 22 shows a schematic representation of another example of the medical device 205. The medical device 205 can be attached to a device 1805 that can be attached to a patient 1800. The device 1805 may communicate with at least one implanted component of a heart support system (for example, the cardiac support system 110). The device 1805 can be connected to the control unit 300 via a cable 1810. Alternatively, the control unit 300 can be connected to the device 1805 via a wireless connection.

The control unit 300 can include an actuator 1815 and a display device 1820. The actuator 1815 can be a key insert or a button that can, for example, turn the control unit 300 on or off. The display unit 1820 can be a touch-sensitive display.

In some implementations, the medical device 205 can include an indicator including at least one light-emitting diode (LED) that can visualize (for example, generate light in different colors, or blink in different patterns) an energy storage status of the storage unit 305. Additionally, the indicator can indicate status or operation of the control unit 300. For example, the indicator of the medical device 205 can represent a supply of electricity (for example, current) to a heart pump.

The control unit 300 can receive power from the storage unit 305. The control unit 300 and the storage unit 305 can be connected via a plug connection (or friction fit), which can make it easier to replace batteries. By reducing the number of cables (for example, by using a wireless plug connection between control unit 300 and memory unit 305), battery changes are simplified. Additionally, the risk of damages to cables or hardware can be reduced. Additionally, eliminating or reducing the number of cables can reduce the risk of patients tripping over cables.

In some implementations, the medical device 205 can establish communication (wired or wireless) with other devices (for example, the device 1805). The medical device 205 can be mounted (or stowed) to the mounting device 200 and receive charge from an external power source (for example, a standard wall plug) or a power supply unit (for example, the storage unit 305) connected to the mounting device 200.

FIG. 23 shows a schematic representation of another example of the medical device 205. The medical device 205 can include the control units 300A, 300B. The medical device 205 can include more than two control units. The control unit 300A can be coupled to the control unit 300B as a separate control unit.

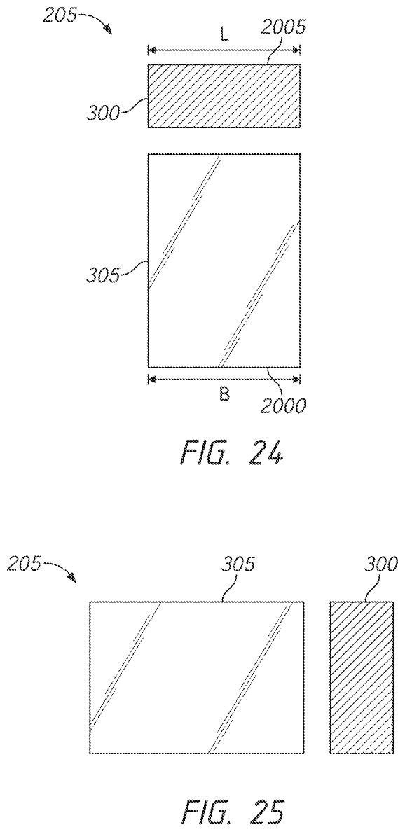

FIG. 24 shows a schematic representation of an example of the medical device 205. As shown in FIG. 24, both the control unit 300 and the memory unit 305 can be rectangular. A side 2000 of the memory unit 305 can have a width B which corresponds to a length L of a side 2005 of the control unit 300. The side 2005 of the control unit 300 can be connected to the side 2000 of the memory unit 305.

The storage unit 305 can be vertically connected to the control unit 300 (for example, connected to the bottom of the control unit 300). Alternatively, as shown in FIG. 25, the control unit 300 can be connected horizontally.

In some implementations, the control unit 300 and the memory unit 305 can be in different shapes (circle, triangle, oval, and so forth) and as such, the control unit 300 can be attached to the storage unit 305 in orientations shown and described herein.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. An apparatus for holding at least one extracorporeal device of a cardiac-support system, the apparatus comprising:
a receiving unit configured to receive at least one extracorporeal device, wherein the at least one extracorporeal device is connected to an implant device of a cardiac support system via a supply line; and
a fastening unit configured to fasten the receiving unit to a furniture.

2. The apparatus of claim 1, wherein the at least one extracorporeal device is a control unit of the cardiac support system or an energy storage unit of the cardiac support system.

3. The apparatus of claim 1, wherein the receiving unit comprises a coupling element configured to couple the at least one extracorporeal device to the receiving unit.

4. The apparatus of claim 1, wherein the receiving unit is configured to receive at least two extracorporeal devices separately from each other.

5. The apparatus of claim 1, wherein the receiving unit is detachably connected to the fastening unit.

6. The apparatus of claim 1, further comprising a cable retention device configured to guide the supply line at least in sections.

7. The apparatus of claim 6, further comprising a power supply unit configured to provide electrical energy to the at least one extracorporeal device.

8. The apparatus of claim 7, further comprising a charging cable configured to provide the electrical energy from the power supply unit to at least one extracorporeal device.

9. The apparatus of claim 7, wherein the power supply unit is configured to wirelessly transmit the electrical energy to the at least one extracorporeal device.

10. The apparatus of claim 1, wherein the fastening unit is configured to connect to the furniture in a form-fitting, non-positive, or material-fitting manner.

11. The apparatus of claim 1, further comprising a cable guide configured to guide a cable extending between an external power supply and the receiving unit.

12. A mounting device for coupling a medical device to a holding device comprising:
a connecting device; and
a receiving hook attached to the connecting device and configured to couple with a medical device.

13. The device of claim 12, wherein the connecting device comprises a cable winding section configured to receive a wound cable and a suspension arm configured to couple the connecting device to a furniture, wherein the suspension arm is positioned adjacent to the cable winding section.

14. The device of claim 12, further comprising a charging device configured to provide an output voltage for the medical device.

15. The device of claim 12, wherein the receiving hook comprises an electrical interface configured to be in contact with a corresponding electrical interface of the medical device when the medical device is coupled with the receiving hook.

16. The device of claim 15, further comprising a charging device, wherein the contact with the electrical interface of the receiving hook and the corresponding electrical interface of the medical device is configured to allow the charging device to provide charge for the medical device.

17. The device of claim 12, further comprising an adapter member configured to couple to the connecting device and the medical device so that the adapter member is positioned between the connecting device and the medical device, wherein the adapter member is configured to establish electrical communication between the connecting device and the medical device.
